(19) United States
(12) Patent Application Publication
(10) Pub. No.: US 2026/0028404 A1
(43) Pub. Date: Jan. 29, 2026

(54) METHODS FOR TREATING CANCER INCLUDING GLATIRAMER ACETATE AND IMMUNE CHECKPOINT INHIBITORS

(71) Applicants: UNIVERSITY OF KANSAS, Lawrence, KS (US); KING ABDULAZIZ UNIVERSITY, Jeddah, Hejaz (SA)

(72) Inventors: Cory BERKLAND, Wildwood, MO (US); Marcus Laird FORREST, Lawrence, KS (US); Nabil Abdulhafiz ALHAKAMY, Jeddah (SA); Aric HUANG, Lawrence, KS (US)

(73) Assignees: UNIVERSITY OF KANSAS, Lawrence, KS (US); KING ABDULAZIZ UNIVERSITY, Jeddah, Hejaz (SA)

(21) Appl. No.: 18/995,466

(22) PCT Filed: Jul. 17, 2023
(86) PCT No.: PCT/US2023/027914

§ 371 Date: Jan. 16, 2025

Related U.S. Application Data
(60) Provisional application No. 63/390,248, filed on Jul. 18, 2022.

(51) Int. Cl. C07K 16/28; A61K 38/02; A61P 35/00

(52) U.S. Cl. CPC C07K 16/2827; A61K 38/02; A61P 35/00; C07K 16/2818

(57) ABSTRACT

The present disclosure relates to methods for treating cancer/inhibiting tumor growth in a patient in need thereof comprising administering to the patient an effective amount of Glatiramer acetate (GA) and an effective amount of an immune checkpoint inhibitor.

### CROSS-REFERENCE TO RELATED APPLICATIONS

This application claims the benefit of and priority to U.S. Provisional Appl. No. 63/390,248, filed Jul. 18, 2022, the disclosure of which is incorporated by reference herein in its entirety.

### TECHNICAL FIELD

The present disclosure relates to methods for treating cancer in a patient in need thereof comprising administering to the patient an effective amount of Glatiramer acetate (GA) and an immune checkpoint inhibitor via intratumoral injection.

### BACKGROUND

The following description of the background of the present technology is provided simply as an aid in understanding the present technology and is not admitted to describe or constitute prior art to the present technology.

The concept of cancer immunosurveillance ascribes a role of cellular immunity in eliminating transformed cells with cytotoxic CD8⁺ T cells being a primary mediator. Cancer cells can express neoantigens to prime conventional CD8⁺ T cells which nonetheless transition to a dysfunctional state of exhaustion characterized by high expression of the immune checkpoint co-inhibitory receptor PD-1 (McLane, et al. Annu Rev Immunol 37, 457-495 (2019)). Despite the clinical success of anti-PD-1 to revive cancer immunity, many patients do not respond to checkpoint blockade therapies. See Sharma, P. & Allison, J. P. Science 348, 56-61 (2015); Topalian, S. L. et al., Nature reviews. Cancer 16, 275-287 (2016); Baumeister, S. H. et al., Annu Rev Immunol 34, 539-573 (2016).

### SUMMARY OF THE PRESENT TECHNOLOGY

In one aspect, the present disclosure provides a method for treating cancer or inhibiting tumor growth in a patient in need thereof comprising administering to the patient an effective amount of GA and an effective amount of an immune checkpoint inhibitor. The GA may be administered separately, simultaneously, or sequentially with the immune checkpoint inhibitor. In some embodiments, GA is administered intratumorally and/or is complexed with CpG oligodeoxynucleotides. Additionally or alternatively, in some embodiments, the immune checkpoint inhibitor is administered intramuscularly, intraperitoneally, subcutaneously, intravenously, or systemically. Examples of cancer include, but are not limited to adrenal cancer, bladder cancer, bone cancer, brain cancer, breast cancer, carcinoma, cervical cancer, colorectal cancer, uterine cancers, ear, nose and throat (ENT) cancers, endometrial cancer, esophageal cancer, gastrointestinal cancer, head and neck cancer, intestinal cancer, kidney cancer, larynx cancer, liver cancer, lung cancer, melanoma, mesothelioma, nasopharynx cancer, neuroblastoma, oral cancer, ovarian cancer, pancreatic cancer, penile cancer, pharynx cancer, prostate cancer, teratomas, testicular cancer, thyroid cancer, vaginal cancers, or vascular tumors. In certain embodiments, the patient is human.

Additionally or alternatively, in some embodiments, GA comprises a heterogenous mixture of copolymers consisting of L-glutamic acid, L-alanine, L-tyrosine, and L-lysine. In certain embodiments, L-glutamic acid, L-alanine, L-tyrosine, and L-lysine are present in an approximate molar ratio of 0.14:0.43:0.09:0.34. In any of the preceding embodiments, GA has a molecular weight ranging from 2,500 to 20,000 Da, or an average molecular weight falling between 5,000 and 9,000 Da.

In some embodiments, the immune checkpoint inhibitor comprises one or more of an anti-PD-1 antibody, an anti-PD-L1 antibody, an anti-PD-L2 antibody, an anti-CTLA-4 antibody, an anti-TIM3 antibody, an anti-CD73 antibody, an or an anti-LAG-3 antibody. Examples of immune checkpoint inhibitors include, but are not limited to pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IBI308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, or BMS-986189.

Also disclosed herein are kits for treating cancer comprising GA, an immune checkpoint inhibitor disclosed herein, and instructions for treating cancer. In some embodiments, GA is complexed with CpG oligodeoxynucleotides.

### BRIEF DESCRIPTION OF THE DRAWINGS

FIG. 1. Representative images used for pre-treatment and post-treatment GeoMx analysis.

FIG. 2. Visual representation for RNAseq gene ontology.

FIG. 3. GA alone increases tumor size (Pressnall et al., International Journal of Pharmaceutics, Volume 605, 10 Aug. 2021, 120812. Two-way ANOVA: p<0.0001 for treatment time and interaction for mannitol vs CpG, R4, and R6; Dunnett post-test vs mannitol *p<0.05, **p<0.01, ***p<0.0001; Dunneett post-test vs CpG #p<0.05, #### p<0.0001, n=5−6.

FIG. 4. Investigating GA in AT84 tumors. Two-way ANOVA with Dunnett's multiple comparison test vs mannitol; *p<0.05, **p<0.01, ****p<0.0001, n=5.

FIG. 5. Tumor growth response curves for the CT26 study. Two-way ANOVA with Tukey's multiple comparison test; ****p<0.0001 vs Iso Man; †p<0.05, ††p<0.01 vs PD1 Man; ‡‡p<0.01 Iso vs PD1 n=3−5.

FIG. 6. IHC analysis for the CT26 study.

FIG. 7. Analysis of systemic cytokine levels in the CT26 tumor model treated with anti-PD-1 therapy in combination with intratumoral GA. Two-way ANOVA with Sidak's multiple comparison test; *p<0.05, **p<0.01, ***p<0.001, ****p<0.0001 vs respective mannitol groups; ††p<0.01, †††p<0.001, ††††p<0.0001 vs respective isotype group; n=4−5.

FIG. 8. E0771 dose escalation with 100 μg anti-PD1.

FIG. 9. E0771 dose escalation with 250 μg anti-PD1.

FIG. 10. Kaplan Meier Survival for the Cloudman M3 study.

FIG. 11. Individual Tumor Growth Curves for the Cloudman M3 study.

FIGS. 12A-12B. Anti-PD-1 therapy in AT84 tumor model reduced lung metastasis when administered in combination with intratumoral CpG or GA-CpG treatment.

FIG. 12A: Representative images of H&E staining of lungs. FIG. 12B: Overview of lung metastasis scoring. 0=absent or no evident of tumor, 0.5=suspect of metastasis (unclear if inflammation or tumors), 1=minimal metastasis, 2=mild, 3=moderate, and 4=severe. Scale bars are shown at 60 μm. Two-way ANOVA with Sidak's multiple comparison test; **p<0.01,n=5−10.

FIG. 13. Female DBA/2N mice were implanted with CloneM3 (Z1) cells into the left mammary fat pad on Day 0 and treated as depicted in the figure legend. Data are displayed as means +/−SEM. The number of animals alive on Day 0 (implantation) and on Day 26 (end of study) are presented in parenthesis for each group in the legend.

FIG. 14. CT26wt primary tumor growth (mm³), displayed for the following Groups: (a) Vehicle Control (25 μl/mouse) i.t. on Days 6, 9, 12, 15 and 18+ Isotype Control (10 mg/kg; 5 ml/kg) i.p. on Days 6, 9 and 13 (n=8/0); (b) GA compound (1 mg; 25 μl/mouse) i.t. on Days 6, 9, 12, 15 and 18+ Isotype Control (10 mg/kg; 5 ml/kg) i.p. on Days 6, 9 and 13 (n=8/0); (c) Vehicle Control (25 μl/mouse) i.t. on Days 6, 9, 12, 15 and 18+ anti-mPD-L1(10 mg/kg; 5 ml/kg) i.p. on Days 6, 9 and 13 (n=8/0); and (d) GA compound (1 mg; 25 μl/mouse) i.t. on Days 6, 9, 12, 15 and 18+ anti-mPD-L1 (10 mg/kg; 5 ml/kg) i.p. on Days 6, 9 and 13 (n=8/2). Female BALB/c mice were implanted with CT26wt tumor cells into the mammary fat pad on Day 0 and were treated as depicted in the figure legends. Primary tumor volumes are displayed as mean values +/−SEM until Day 17. The number of animals alive on Days 0 (implantation) and 45 (study termination), are shown for each group in parenthesis.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

The present disclosure demonstrates that combination therapy with glatiramer acetate and an immune checkpoint inhibitor are useful in methods for treating cancer/inhibiting tumor growth in a patient in need thereof.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value). If there are uses of the term which are not clear to persons of ordinary skill in the art, given the context in which it is used, “about” will mean up to plus or minus 10% of the particular term—for example, “about 10 wt. %” would be understood to mean “9 wt. % to 11 wt. %.” It is to be understood that when “about” precedes a term, the term is to be construed as disclosing “about” the term as well as the term without modification by “about”—for example, “about 10 wt. %” discloses “9 wt. % to 11 wt. %” as well as disclosing “10 wt. %.”

The phrase “and/or” as used herein will be understood to mean any one of the recited members individually or a combination of any two or more thereof—for example, “A, B, and/or C” would mean “A, B, C, A and B, A and C, B and C, or the combination of A, B, and C.”

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

“Immune checkpoint inhibitor(s)” as used herein refers to molecules that completely or partially reduce, inhibit, interfere with or modulate the activity of one or more checkpoint proteins. Checkpoint proteins regulate T-cell activation or function. Checkpoint proteins include, but are not limited to CTLA-4 and its ligands CD80 and CD86; PD-1 and its ligands PDL1 and PDL2; LAGS, B7-H3, B7-H4, TIM3, ICOS, and BTLA (Pardoll et al. Nature Reviews Cancer 12:252-264 (2012)).

As used herein, “intratumoral” includes regions within and/or around tumor tissues (e.g., “near to” tumor tissues), and thus encompasses intralesional, peritumoral, perilesional, etc. delivery. As used herein, “intratumoral injection” and “administered intratumorally” includes injection within the outer perimeter of the tumor, injections “near to” a tumor that access the same network of draining lymph nodes, or both. “Near to” will be understood by persons of ordinary skill in the art and will vary to some extent depending upon the context in which it is used. If there are uses of the term which are not clear to persons of ordinary skill in the art, given the context in which it is used, “near to” a tumor will mean no more than about 10 millimeters from an outer perimeter of the tumor; thus, “near to” a tumor would be understood to mean a distance from an outer perimeter of the tumor of about 10 millimeters, about 9 millimeters, about 8 millimeters, about 7 millimeters, about 6 millimeters, about 5 millimeters, about 4 millimeters, about 3 millimeters, about 2 millimeters, about 1 millimeters, about 0.5 millimeters, about 0.01 millimeters, or any range including and/or in between any two of these values.

As used herein, the terms “individual”, “patient”, or “subject” can be an individual organism, a vertebrate, a mammal, or a human. In some embodiments, the individual, patient or subject is a human.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, the term “therapeutic agent” is intended to mean a compound that, when present in an effective amount, produces a desired therapeutic effect on a subject in need thereof.

As used herein, “prevention” or “preventing” of a disorder or condition refers to a compound that, in a statistical sample, reduces the occurrence of the disorder or condition in the treated sample relative to an untreated control sample, or delays the onset of one or more symptoms of the disorder or condition relative to the untreated control sample.

As used herein, the term “therapeutic agent” is intended to mean a compound that, when present in an effective amount, produces a desired therapeutic effect on a subject in need thereof.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

### Glatiramer Acetate (GA)

Glatiramer acetate (GA), which is the active ingredient in CopaxoneR, is one of the most widely prescribed treatments for relapsing-remitting multiple sclerosis (RRMS). GA was originally designed as a myelin basic protein (MBP) mimic to induce experimental autoimmune encephalomyelitis (EAE) in mice; however, GA was found to suppress EAE instead. GA is a heterogenous mixture of copolymers consisting of L-glutamic acid, L-alanine, L-tyrosine, and L-lysine in an approximate molar ratio of 0.14:0.43:0.09:0.34. It has a molecular weight ranging from 2,500 to 20,000 Da, with the average falling between 5,000 and 9,000 Da. The isoelectric point (pI) of MBP is estimated to be around 10, and since GA is a mimic of MBP, GA is expected to have a large net positive charge.

Pharmacokinetic reports of GA are varied; however, current understanding suggests GA is primarily localized at the site of injection. The physicochemical behavior of GA was studied in vitro and in vivo (Song et al., J Control Release. 2019 Jan. 10;293:36-47). The structure of GA is defined as a function of pH and temperature. Subsequent studies utilized hyaluronic acid (HA) to simulate the subcutaneous injection site. GA was observed to form aggregates and precipitate in the presence of HA. Studies in mice confirmed GA also formed spherical aggregates near the injection site and the aggregates associated with glycosaminoglycans in the extracellular matrix.

### Pharmaceutical Compositions Including Immune Checkpoint Inhibitors and/or GA

The pharmaceutical compositions of the present technology can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions. Formulations may optionally contain solvents, diluents, and other liquid vehicles, dispersion or suspension aids, surface active agents, pH modifiers, isotonic agents, thickening or emulsifying agents, stabilizers and preservatives, solid binders, lubricants and the like, as suited to the particular dosage form desired. In certain embodiments, the compositions disclosed herein are formulated for administration to a mammal, such as a human.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, cyclodextrins, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. Compositions formulated for parenteral administration may be injected by bolus injection or by timed push, or may be administered by continuous infusion.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents such as phosphates or carbonates.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. They may optionally contain opacifying agents and can also be of a composition that they release the active ingredient(s) only, or in a certain part of the intestinal tract, optionally, in a delayed manner. Examples of embedding compositions that can be used include polymeric substances and waxes.

The active compounds can also be in micro-encapsulated form with one or more excipients as noted above. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or in a certain part of the intestinal tract, optionally, in a delayed manner. Examples of embedding compositions that can be used include polymeric substances and waxes.

### Modes of Administration and Effective Dosages

Any method known to those in the art for contacting a cell, organ or tissue with one or more immune checkpoint inhibitors and/or GA disclosed herein may be employed. Suitable methods include in vitro, ex vivo, or in vivo methods. In vivo methods typically include the administration of one or more immune checkpoint inhibitors and/or GA to a mammal, suitably a human. When used in vivo for therapy, the one or more immune checkpoint inhibitors and/or GA described herein are administered to the subject in effective amounts (i.e., amounts that have desired therapeutic effect). The dose and dosage regimen will depend upon the degree of the disease state of the subject, the characteristics of the particular immune checkpoint inhibitor or GA used, e.g., its therapeutic index, and the subject's history.

The effective amount may be determined during pre-clinical trials and clinical trials by methods familiar to physicians and clinicians. An effective amount of one or more immune checkpoint inhibitors and/or GA useful in the methods may be administered to a mammal in need thereof by any of a number of well-known methods for administering pharmaceutical compounds. The immune checkpoint inhibitor or GA may be administered systemically or locally.

The one or more immune checkpoint inhibitors and/or GA described herein can be incorporated into pharmaceutical compositions for administration, singly or in combination, to a subject for the treatment or prevention of a disease or condition described herein. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., 7 days of treatment).

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The pharmaceutical compositions having one or more immune checkpoint inhibitors and/or GA disclosed herein can include a carrier, which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressurized container or dispenser, which contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

A therapeutic agent can be formulated in a carrier system. The carrier can be a colloidal system. The colloidal system can be a liposome, a phospholipid bilayer vehicle. In one embodiment, the therapeutic agent is encapsulated in a liposome while maintaining the agent's structural integrity. One skilled in the art would appreciate that there are a variety of methods to prepare liposomes. (See Lichtenberg, et al., Methods Biochem. Anal., 33:337-462(1988); Anselem, et al., Liposome Technology, CRC Press (1993)). Liposomal formulations can delay clearance and increase cellular uptake (See Reddy, Ann. Pharmacother., 34 (7-8):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic agent can be embedded in the polymer matrix, while maintaining the agent's structural integrity. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compounds can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,” Trends Biotechnol., 13(12):527-37 (1995). Mizguchi, et al., Cancer Lett., 100:63-69 (1996), describes the use of fusogenic liposomes to deliver a protein to cells both in vivo and in vitro.

Dosage, toxicity and therapeutic efficacy of any therapeutic agent can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately. Levels in plasma may be measured, for example, by high performance liquid chromatography.

Typically, an effective amount of the one or more immune checkpoint inhibitors and/or GA disclosed herein sufficient for achieving a therapeutic or prophylactic effect, range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example, dosages can be 1 mg/kg body weight or 10 mg/kg body weight every day, every two days or every three days or within the range of 1-10 mg/kg every week, every two weeks or every three weeks. In one embodiment, a single dosage of the therapeutic compound ranges from 0.001-10,000 micrograms per kg body weight. In one embodiment, one or more immune checkpoint inhibitor/GA concentrations in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

In some embodiments, a therapeutically effective amount of one or more immune checkpoint inhibitors and/or GA may be defined as a concentration of the agent at the target tissue of 10⁻³² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., parenteral infusion or transdermal application).

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance with the present methods can be any mammal, including, for example, farm animals, such as sheep, pigs, cows, and horses; pet animals, such as dogs and cats; laboratory animals, such as rats, mice and rabbits. In some embodiments, the mammal is a human.

### Therapeutic Methods of the Present Technology

In one aspect, the present disclosure provides a method for treating cancer or inhibiting tumor growth in a patient in need thereof comprising administering to the patient an effective amount of GA and an effective amount of an immune checkpoint inhibitor. The GA may be administered separately, simultaneously, or sequentially with the immune checkpoint inhibitor. In some embodiments, GA is administered intratumorally and/or is complexed with CpG oligodeoxynucleotides. Additionally or alternatively, in some embodiments, the immune checkpoint inhibitor is administered intramuscularly, intraperitoneally, subcutaneously, intravenously, or systemically. Examples of cancer include, but are not limited to adrenal cancer, bladder cancer, bone cancer, brain cancer, breast cancer, carcinoma, cervical cancer, colorectal cancer, uterine cancers, ear, nose and throat (ENT) cancers, endometrial cancer, esophageal cancer, gastrointestinal cancer, head and neck cancer, intestinal cancer, kidney cancer, larynx cancer, liver cancer, lung cancer, melanoma, mesothelioma, nasopharynx cancer, neuroblastoma, oral cancer, ovarian cancer, pancreatic cancer, penile cancer, pharynx cancer, prostate cancer, teratomas, testicular cancer, thyroid cancer, vaginal cancers, or vascular tumors. In certain embodiments, the patient is human.

Additionally or alternatively, in some embodiments, GA comprises a heterogenous mixture of copolymers consisting of L-glutamic acid, L-alanine, L-tyrosine, and L-lysine. In certain embodiments, L-glutamic acid, L-alanine, L-tyrosine, and L-lysine are present in an approximate molar ratio of 0.14:0.43:0.09:0.34. In any of the preceding embodiments, GA has a molecular weight ranging from 2,500 to 20,000 Da, or an average molecular weight falling between 5,000 and 9,000 Da.

In some embodiments, the immune checkpoint inhibitor comprises one or more of an anti-PD-1 antibody, an anti-PD-L1 antibody, an anti-PD-L2 antibody, an anti-CTLA-4 antibody, an anti-TIM3 antibody, an anti-CD73 antibody, an or an anti-LAG-3 antibody. Examples of immune checkpoint inhibitors include, but are not limited to pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IBI308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, or BMS-986189.

Additionally or alternatively, in some embodiments of the methods disclosed herein, the immune checkpoint inhibitor can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), simultaneously with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of GA to a patient with cancer.

In some embodiments, the immune checkpoint inhibitor and GA are administered to a patient, for example, a mammal, such as a human, in a sequence and within a time interval such that the inhibitor that is administered first acts together with the inhibitor that is administered second to provide greater benefit than if each inhibitor were administered alone. For example, the immune checkpoint inhibitor and GA can be administered at the same time or sequentially in any order at different points in time; however, if not administered at the same time, the immune checkpoint inhibitor and GA are administered sufficiently close in time so as to provide the desired therapeutic or prophylactic effect of the combination of the two inhibitors. In one embodiment, the immune checkpoint inhibitor and GA exert their effects at times which overlap. In some embodiments, the immune checkpoint inhibitor and GA each are administered as separate dosage forms, in any appropriate form and by any suitable route. In other embodiments, the immune checkpoint inhibitor and GA are administered simultaneously in a single dosage form.

It will be appreciated that the frequency with which any of these therapeutic agents can be administered can be once or more than once over a period of about 2 days, about 3 days, about 4 days, about 5 days, about 6 days, about 7 days, about 8 days, about 9 days, about 10 days, about 11 days, about 12 days, about 13 days, about 14 days, about 20 days, about 28 days, about a week, about 2 weeks, about 3 weeks, about 4 weeks, about a month, about every 2 months, about every 3 months, about every 4 months, about every 5 months, about every 6 months, about every 7 months, about every 8 months, about every 9 months, about every 10 months, about every 11 months, about every year, about every 2 years, about every 3 years, about every 4 years, or about every 5 years.

For example, an immune checkpoint inhibitor or GA may be administered daily, weekly, biweekly, or monthly for a particular period of time. An immune checkpoint inhibitor or GA may be dosed daily over a 14 day time period, or twice daily over a seven day time period. An immune checkpoint inhibitor or GA may be administered daily for 7 days.

Alternatively, an immune checkpoint inhibitor or GA may be administered daily, weekly, biweekly, or monthly for a particular period of time followed by a particular period of non-treatment. In some embodiments, the immune checkpoint inhibitor or GA can be administered daily for 14 days followed by seven days of non-treatment, and repeated for two more cycles of daily administration for 14 days followed by seven days of non-treatment. In some embodiments, the immune checkpoint inhibitor or GA can be administered twice daily for seven days followed by 14 days of non-treatment, which may be repeated for one or two more cycles of twice daily administration for seven days followed by 14 days of non-treatment.

In some embodiments, the immune checkpoint inhibitor or GA is administered daily over a period of 14 days. In another embodiment, the immune checkpoint inhibitor or GA is administered daily over a period of 12 days, or 11 days, or 10 days, or nine days, or eight days. In another embodiment, the immune checkpoint inhibitor or GA is administered daily over a period of seven days. In another embodiment, the immune checkpoint inhibitor or GA is administered daily over a period of six days, or five days, or four days, or three days.

In some embodiments, individual doses of the immune checkpoint inhibitor and the GA are administered within a time interval such that the two inhibitors can work together (e.g., within 1 hour, 2 hours, 4 hours, 6 hours, 12 hours, 24 hours, 48 hours, 72 hours, 96 hours, 5 days, 6 days, 1 week, or 2 weeks). In some embodiments, the treatment period during which the therapeutic agents are administered is then followed by a non-treatment period of a particular time duration, during which the therapeutic agents are not administered to the patient. This non-treatment period can then be followed by a series of subsequent treatment and non-treatment periods of the same or different frequencies for the same or different lengths of time. In some embodiments, the treatment and non-treatment periods are alternated. It will be understood that the period of treatment in cycling therapy may continue until the patient has achieved a complete response or a partial response, at which point the treatment may be stopped. Alternatively, the period of treatment in cycling therapy may continue until the patient has achieved a complete response or a partial response, at which point the period of treatment may continue for a particular number of cycles. In some embodiments, the length of the period of treatment may be a particular number of cycles, regardless of patient response. In some other embodiments, the length of the period of treatment may continue until the patient relapses.

In some embodiments, the immune checkpoint inhibitor and the GA are cyclically administered to a patient. Cycling therapy involves the administration of a first agent (e.g., a first prophylactic or therapeutic agent) for a period of time, followed by the administration of a second agent and/or third agent (e.g., a second and/or third prophylactic or therapeutic agent) for a period of time and repeating this sequential administration. Cycling therapy can reduce the development of resistance to one or more of the therapies, avoid or reduce the side effects of one of the therapies, and/or improve the efficacy of the treatment.

In some embodiments, the immune checkpoint inhibitor is administered for a particular length of time prior to administration of the GA. For example, in a 21-day cycle, the immune checkpoint inhibitor may be administered on days 1 to 5, days 1 to 7, days 1 to 10, or days 1 to 14, and the GA may be administered on days 6 to 21, days 8 to 21, days 11 to 21, or days 15 to 21. In other embodiments, the GA is administered for a particular length of time prior to administration of the immune checkpoint inhibitor. For example, in a 21-day cycle, the GA may be administered on days 1 to 5, days 1 to 7, days 1 to 10, or days 1 to 14, and the immune checkpoint inhibitor may be administered on days 6 to 21, days 8 to 21, days 11 to 21, or days 15 to 21.

In one embodiment, the administration is on a 21-day dose schedule in which a once daily dose of immune checkpoint inhibitor is administered beginning on day eight for seven days, followed by seven days of non-treatment, in combination with twice-daily administration of the GA for seven days followed by 14 days of non-treatment (e.g., the immune checkpoint inhibitor is administered on days 8-14 and the GA is administered on days 1-7 of the 21-day schedule). In another embodiment, the administration is on a 21-day dose schedule in which a once daily dose of GA is administered beginning on day eight for seven days, followed by seven days of non-treatment, in combination with twice-daily administration of the immune checkpoint inhibitor for seven days followed by 14 days of non-treatment (e.g., the GA is administered on days 8-14 and the immune checkpoint inhibitor is administered on days 1-7 of the 21-day schedule).

In some embodiments, the immune checkpoint inhibitor and GA each are administered at a dose and schedule typically used for that agent during monotherapy. In other embodiments, when the immune checkpoint inhibitor and GA are administered concomitantly, one or both of the agents can advantageously be administered at a lower dose than typically administered when the agent is used during monotherapy, such that the dose falls below the threshold that an adverse side effect is elicited.

The therapeutically effective amounts or suitable dosages of the immune checkpoint inhibitor and the GA in combination depends upon a number of factors, including the nature of the severity of the condition to be treated, the particular inhibitor, the route of administration and the age, weight, general health, and response of the individual patient. In certain embodiments, the suitable dose level is one that achieves a therapeutic response as measured by tumor regression or other standard measures of disease progression, progression free survival, or overall survival. In other embodiments, the suitable dose level is one that achieves this therapeutic response and also minimizes any side effects associated with the administration of the therapeutic agent.

Suitable daily dosages of immune checkpoint inhibitors can generally range, in single or divided or multiple doses, from about 10% to about 120% of the maximum tolerated dose as a single agent. In certain embodiments, the suitable dosages of immune checkpoint inhibitors are from about 20% to about 100% of the maximum tolerated dose as a single agent. In other embodiments, the suitable dosages of immune checkpoint inhibitors are from about 25% to about 90% of the maximum tolerated dose as a single agent. In some embodiments, the suitable dosages of immune checkpoint inhibitors are from about 30% to about 80% of the maximum tolerated dose as a single agent. In other embodiments, the suitable dosages of immune checkpoint inhibitors are from about 40% to about 75% of the maximum tolerated dose as a single agent. In some embodiments, the suitable dosages of immune checkpoint inhibitors are from about 45% to about 60% of the maximum tolerated dose as a single agent. In other embodiments, suitable dosages of immune checkpoint inhibitors are about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, or about 120% of the maximum tolerated dose as a single agent.

Suitable daily dosages of GA can generally range, in single or divided or multiple doses, from about 10% to about 120% of the maximum tolerated dose as a single agent. In certain embodiments, the suitable dosages of GA are from about 20% to about 100% of the maximum tolerated dose as a single agent. In some other embodiments, the suitable dosages of GA are from about 25% to about 90% of the maximum tolerated dose as a single agent. In some other embodiments, the suitable dosages of GA are from about 30% to about 80% of the maximum tolerated dose as a single agent. In some other embodiments, the suitable dosages of GA are from about 40% to about 75% of the maximum tolerated dose as a single agent. In some other embodiments, the suitable dosages of GA are from about 45% to about 60% of the maximum tolerated dose as a single agent. In other embodiments, suitable dosages of GA are about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, or about 120% of the maximum tolerated dose as a single agent.

### Kits

The present disclosure provides kits for treating cancer comprising GA, an immune checkpoint inhibitor disclosed herein, and instructions for treating cancer. When simultaneous administration is contemplated, the kit may comprise GA and an immune checkpoint inhibitor that has been formulated into a single pharmaceutical composition such as a tablet, or as separate pharmaceutical compositions. When the GA and the immune checkpoint inhibitor are not administered simultaneously, the kit may comprise GA and an immune checkpoint inhibitor that has been formulated as separate pharmaceutical compositions either in a single package, or in separate packages.

The kits may further comprise pharmaceutically acceptable excipients, diluents, or carriers that are compatible with one or more kit components described herein. Optionally, the above described components of the kits of the present technology are packed in suitable containers and labeled for the treatment of cancer. The kits may optionally include instructions customarily included in commercial packages of therapeutic products, that contain information about, for example, the indications, usage, dosage, manufacture, administration, contraindications and/or warnings concerning the use of such therapeutic products.

### EXAMPLES

The present technology is further illustrated by the following Examples, which should not be construed as limiting in any way.

### Example 1: Experimental Methods and Materials

Clinical trial: A single-arm, open-label, preoperative window of opportunity trial was conducted to evaluate the feasibility, safety, and local effects of intratumoral/peritumoral injections of Glatiramer acetate (GA) (Copaxone®). Subjects whose tumors were accessible for intratumoral, percutaneous injection and were planned for surgery as the primary treatment were eligible if no other treatment between initial biopsy and surgery was anticipated. During the treatment period, eligible subjects received Copaxone® at 40 mg intratumorally for up to 3 times a week and at least 48 hours apart until 24 hours before the planned surgery. This dosing regimen was consistent with the label for subcutaneous injections of Copaxone®.

This study is an open label single arm window of opportunity trial in patients with resectable solid malignancies whose planned primary treatment is surgical resection conducted at the University of Kansas Cancer Center (KUCC). The patients had to be at least 18 years old with previously untreated histologically confirmed malignant tumor that is percutaneously accessible for intratumoral injection. The injectable tumor must be at least 5 mm in diameter using standard measuring tape. Pretreatment archival tumor tissue consisting of formalin-fixed, paraffin-embedded (FFPE) tumor tissue obtained from standard of care diagnostic biopsy must be available in order to be eligible for the trial. Patients whose lesions are in close proximity to vascular structures (i.e., carotid artery or tumors close to other vital organs such as the trachea), with mucosal lesions only, with known hypersensitivity to Copaxone or with a known condition that leads to immunosuppression such as AIDS or concurrent use of immunosuppressive therapy were considered ineligible. Approval for the study was granted through the institutional review board at University of Kansas Cancer Center and all patients provided written informed consent before study entry. This trial was performed according to the Declaration of Helsinki principles. The KUCC Data and Safety Monitoring Committee (DSMC) performed the oversight of the monitoring of participant safety, conduct and scientific progress of research protocols, and the validity and integrity of the data for clinical trials.

Patients with palpable head and neck tumors who progressed to a scheduled resection were eligible to participate in this window of opportunity trial. Biopsies of tumor tissues were compared to resected tumors that had been treated with 1, 2, or 3 Copaxone® injections. Safety observations were made, and paired biopsy and resection samples were analyzed using traditional histological staining, RNAseq, and spatial cancer transcriptome analysis (i.e., Nanostring GeoMx Digital Spatial Profiler) to determine changes in the tumor microenvironment after treatment.

Study treatment: Treatment was administered on an outpatient basis prior to surgery. Glatiramer acetate (Copaxone®) at 40 mg (40 mg/ml) dose was administered by intratumoral injection, for at least 1 dose up to a maximum of 3 doses prior to surgery. Doses were administered at least 48 hours apart and the last dose given within 96 hours of surgery. This window of 96 hours was chosen because we hypothesized that any immune changes due to Copaxone may remain apparent within 96 hours. No dose adjustments, modifications or delays due to Copaxone® related toxicity were allowed.

Injection Technique: After selection of percutaneously accessible tumors the assigned dose of glatiramer acetate (Copaxone®) was administered using a 28-gauge or smaller needle. Efforts were made to administer the full dose into the lesion, unless it was deemed not practicable by the treating investigator. Tumors were injected using a 28-gauge or smaller needle. Glatiramer acetate (Copaxone®) was thoroughly distributed within the injected tumor using a “fanning method” (to distribute the injection across several angles throughout the lesion to maximize the spread of glatiramer acetate (Copaxone®) in the tumor). The use of local anesthetic prior to Copaxone injection was allowed.

Safety Assessments: Adverse events were assessed at baseline, during Copaxone® dose administration and at time of surgery or end of treatment visit. The revised NCI Common Terminology Criteria for Adverse Events (CTCAE) version 5.0 was utilized for AE reporting and toxicity assessment. Complete blood count, chemistry and INR were done at baseline.

GeoMx DSP analysis: Digital spatial profiling of pre-and post-treatment formalin-fixed, paraffin embedded (FFPE) tumor tissue was performed using the GeoMx DSP platform (NanoString Technologies) to measure levels of 28 protein markers associated with immune oncology. Specifically, the immune cell profiling core protein module and IO drug target protein module were used for this analysis following manufacturer's protocol for staining, hybridization, collection, detection (nCounter based-counting), and data normalization. Briefly, this entailed an initial pathologist review of hematoxylin and eosin-stained sections by a pathologist at KUMC to verify presence of tumor cells within the FFPE block. Next, a 5 μm-think unstained FFPE slide was subjected to a cocktail of primary antibodies conjugated with unique DNA-oligonucleotide moieties attached with a light sensitive photocleavable linker. Within this cocktail were also anti-pan-cytokeratin (included with GeoMx assay and used for the basal cell carcinoma and squamous cell carcinoma samples) or anti-MART-1 (Novus Biologicals, catalog #NBP2-46603AF647 used for targeting epithelium in melanoma samples), CD45 (for targeting immune cells) and Syto-13 (used as a nuclear stain). These fluorescently labeled antibodies were used for imaging and identifying the regions of interest (ROIs) followed by segmentation into tumor cells and immune cells for independent collection from each ROI into individual wells of a 96-well plate followed by digital quantification using the NanoString nCounter platform. The GeoMx DSP Analysis Suite version 2.4 was used to perform data QC, normalization, and statistical analysis following guidance by NanoString field scientists. For analysis of similar cell populations (i.e., expression levels only in epithelial cells or only in immune cells), the geometric mean of housekeeping proteins GAPDH, Histone H3, and S6 was used for normalization. When different cell populations were being analyzed (i.e., expression levels in epithelial versus immune cells), the geometric mean of the signal-to-noise ratio of negative control mouse and rabbit isotype IgG controls was used for background correction prior.

RNASeq Analysis: A cutoff linear fold-change of 4 was used to select for the differentially expressed genes in each patient. Gene ontology (GO) of the biological process, functional annotation clustering, and Kyoto Encyclopedia of Genes and Genomes (KEGG) pathway analysis of the differentially expressed genes was performed using Database for Annotation, Visualization, and Integrated Discovery (DAVID) v2021^{7,8}. For groups that had more than 3000 differentially expressed genes (i.e., upregulated gene expression for patient 1 and 3), functional annotation clustering was performed using the top 3000 most differently expressed genes.

Ki-67 and Caspase-3 levels: To potentially detect an early signal for anti-tumoral efficacy of Copaxone® treatment, changes in the level of Ki-67 and caspase-3 before and after treatment with Copaxone® were evaluated by IHC. FFPE tumor sections from pre-and post-treatment specimens were mounted on the same slide and stained using Caspase 3 (Biocare, Pacheco, CA) and Ki-67 (MIB-1) (Dako, Carpinteria, CA).

### Example 2: Analysis of Human Clinical Trial Data

Although cancer immunotherapies hold promise against the most resistant cancers, activating the immune system against primary solid tumors and perhaps even against secondary metastases (i.e. the ‘Abscopal Effect’) will likely require a multipronged approach (Ngwa, W. et al. Using immunotherapy to boost the abscopal effect. Nat Rev Cancer 18, 313-322, doi: 10.1038/nrc.2018.6 (2018)). Immune checkpoint inhibitors ‘release the brakes’ on the immune system and are expected to work synergistically with immunostimulants that ‘hit the gas’ in tumor tissue (Vafaei, S. et al. Combination therapy with immune checkpoint inhibitors (ICIs); a new frontier. Cancer Cell International 22, 2, doi: 10.1186/s12935-021-02407-8 (2022)). In this regard, intratumoral injection of immunostimulants that persist at the injection site are particularly attractive since side effects and interactions with systemic cancer immunotherapies may be mitigated. Multiple cancer clinical trials are currently underway to assess such combination approaches to immunotherapy (Ngwa, W. et al. Using immunotherapy to boost the abscopal effect. Nat Rev Cancer 18, 313-322, doi: 10.1038/nrc.2018.6 (2018); Aznar, M. A. et al. Intratumoral Delivery of Immunotherapy-Act Locally, Think Globally. J. Immunol 198, 31-39, doi: 10.4049/jimmunol. 1601145 (2017)), and the recent acquisitions of Checkmate Pharmaceuticals (Regeneron) and Immune Design (Merck) highlight commercial interest.

Understanding that GA has negligible systemic exposure in humans and having confirmed that GA persists in mouse tumors after intratumoral injection, this clinical study aimed to establish key clinical data for Copaxone® as an HIT-IT.

A single-arm, open-label, preoperative window of opportunity trial was conducted to evaluate the feasibility, safety, and local effects of intratumoral/peritumoral injections of Copaxone®. Subjects whose tumors were accessible for intratumoral, percutaneous injection and were planned for surgery as the primary treatment were eligible if no other treatment between initial biopsy and surgery was anticipated. During the treatment period, eligible subjects received Copaxone® at 40 mg intratumorally for up to 3 times a week and at least 48 hours apart until 24 hours before the planned surgery. This dosing regimen was consistent with the label for subcutaneous injections of Copaxone®. Comprehensive chemistry panel and blood counts were be obtained on Day 1 and on the day of surgery. The primary endpoint was adverse events associated with intratumoral/peritumoral injections of Copaxone®. Injection site reactions were observed in and around tumor tissues, similar to subcutaneous injections of Copaxone® reported in MS patients.

TABLE A
Patient information.

| | | | | Number of |
|---|---|---|---|---|
| | | | | Copaxone |
| | | | | Intratumoral |
| Age | Sex | Race | Diagnosis | Injections |
| 73 | M | White | Basal cell, neck | 2 |
| 64 | M | White | Basal cell cancer, nose | 2 |
| 66 | M | White | Squamous cell cancer, | 1 |
| | | | scalp | |
| 77 | M | White | Basal cell cancer, chest | 3 |
| 43 | F | White | Melanoma, ear | 2 |
| 67 | M | White | melanoma, scalp | 2 |
| 78 | M | White | Squamous cell cancer, | 2 |
| | | | scalp | |
| 65 | M | White | Melanoma, back | 2 |
| 48 | M | White | Melanoma, back | 2 |

Table A shows the patient demographics. Eleven patients were consented for this trial. Nine patients were enrolled with a median age of 66 years (43-78). All but one patient were male (88%). Four patients had melanoma, 3 had basal cell cancer of the skin and 2 had squamous cell cancer of the scalp. One patient had 3 injections of Copaxone, 7 had 2injections, and 1 had 1 injection. The median time from the last Copaxone injection to approximate time of tissue retrieval was 47 hours (range of 23-73 hours). There were only 3treatment related adverse events reported. All were grade 1. One patient with tumor in the surface of the nose had grade 1 injection site reaction described as pressure and itching. Another patient had grade 1 burning with injection of lidocaine. There were no grade 2 or higher adverse events. All treated patients underwent planned surgery.

Immune biomarkers were assessed in tumor biopsies and compared with those in the resected tumors after treatment. Conventional RNA sequencing and a geographical sequencing platform (GeoMx) were used to assess immune markers at the transcriptional level. In the RNA-Seq gene expression analysis, genes were filtered for differentially expressed genes (|FC|<4) between pre-and post-samples for three patients. For patient 1, 6355 genes were upregulated and 406 genes were downregulated post treatment. For patient 2, 647 genes were upregulated and 75 genes were downregulated post treatment. For patient 3, 5375 genes were upregulated and 128 genes were downregulated post treatment.

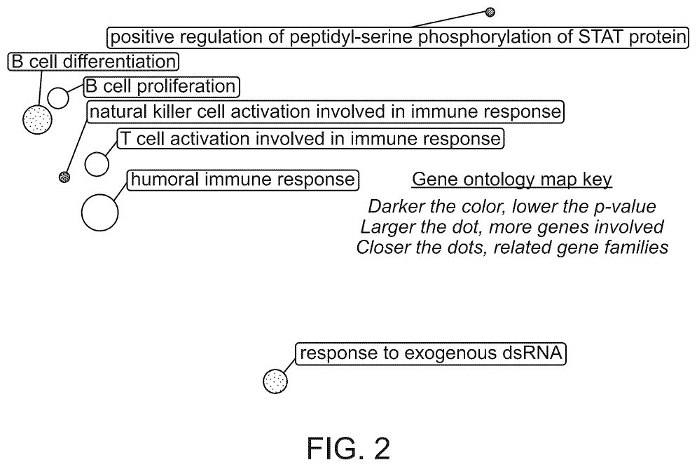

Gene Ontology (GO) enrichment analysis was performed to categorizes differential gene expressions into specific biological processes followed by Functional Annotation Clustering to group related GO terms (see FIG. 2). All three patients had an upregulation in genes involved in promoting an immune-rich tumor microenvironment such as NK cell activation, positive regulation of STAT protein, T-cell activation, humoral immune response, response to exogenous dsRNA, and B-cell proliferation and differentiation. Another study characterized effects of GA on recruiting and activating T cells (including Natural Killer T cells), complimenting the gene expression observations collected here (Maghazachi, A. A., Sand, K. L. & Al-Jaderi, Z. Glatiramer Acetate, Dimethyl Fumarate, and Monomethyl Fumarate Upregulate the Expression of CCR10 on the Surface of Natural Killer Cells and Enhance Their Chemotaxis and Cytotoxicity. Front Immunol 7, 437, doi: 10.3389/fimmu.2016.00437 (2016)). No biological processes were consistently found to be downregulated post treatment.

The GeoMx Digital Spatial Profiling (DSP) platform (NanoString) was used to profile 28 immune related proteins. This platform allows users to perform nondestructive interrogation of formalin-fixed paraffin embedded (FFPE) tissue (among other tissue types) for multiplexed spatial profiling of immunooncology-related proteins to assess tumor cells and their microenvironment. The GeoMx platform uses a cocktail of antibodies conjugated to photocleavable DNA-barcoded oligos to provide high multiplex capacity and the use of guided ultraviolet light exposure (by way of adjustable 1 million micromirrors) provides a high degree of flexibility in selection of regions of interest for study. DSP technology and the GeoMx platform have been extensively reviewed.

Using the GeoMx DSP platform, we performed in situ digital spatial profiling of tissue specimens collected from the COPAX trial patients before and after treatment to measure levels of 28 proteins (Immune cell profiling panel, and the IO drug target panel). After a pathologist review of H&E-stained tissue specimens to assess presence of tumor cells, GeoMx DSP analysis was performed on a serial unstained slide for each sample. Regions of interest (ROIs) which encompassed both tumor and immune cells were selected either by free-hand drawing using the polygon tool or using a standard circle. The ROIs were further segmented into tumor cells and immune cells based on the staining pattern of morphology markers (pan-cytokeratin and CD45 for the carcinomas and MARTI and CD45 for the melanomas). Following segmentation, the DNA bar codes corresponding to each target in the antibody cocktail were collected by the instrument. The DNA bar codes were processed through the nCounter for digital counting and finally analyzed through the GeoMx Analysis suite. Initial data analysis of immune cells and tumor cells in pretreatment tissue showed the expected pattern where most of the immune related targets are expressed at higher levels in the immune cells relative to the tumor cells and the few epithelial targets included in the panels are expressed at higher levels in the tumor cells relative to the immune cells. This initial analysis comparing the two different cell populations serves as a control to verify that the targets are being expressed and measured as expected.

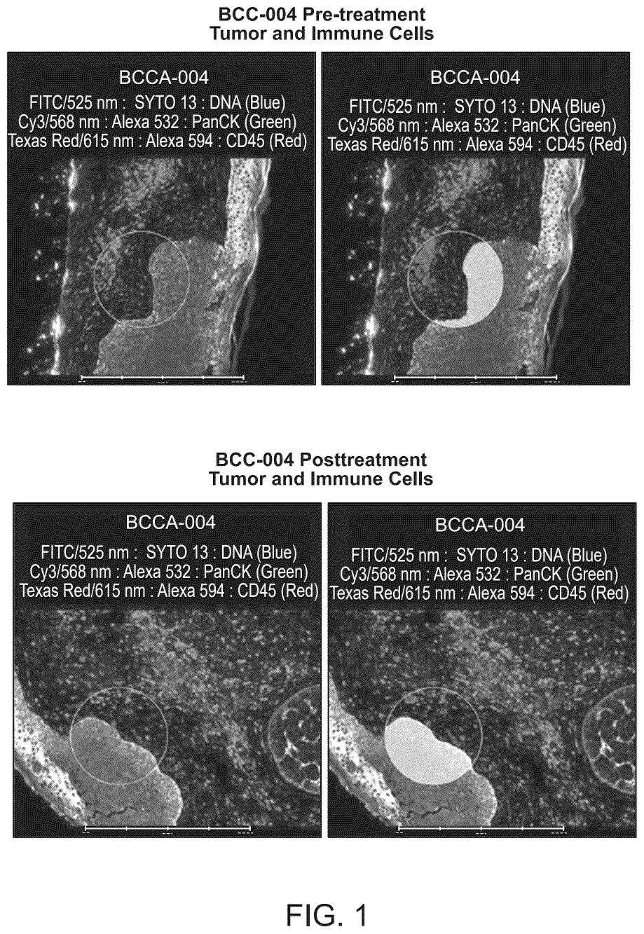

Exemplary images (see FIG. 1) for BCCa tumor biopsy (pre-treatment) and resected tumors (treated) used panCK as tumor marker (green) and CD45 (cyan) as immune cell marker (DNA was stained blue). Regions of interest at the tumor margin were analyzed (circled). Genes were considered upregulated if expression increased >100-fold and were statistically significant across 16 measurements comparing biopsy section to tumor section. (ClinicalTrials.gov Identifier: NCT03982212, BCCa: Basal Cell Carcinoma, SCCa: Squamous Cell Carcinoma). Upregulated genes indicated Copaxone® had an immunostimulatory effect at the intratumoral injection site (see Table B). Several patients exhibited upregulated expression of genes that encode targets for checkpoint inhibitor therapy.

Intratumoral administration of immunostimulants such as pattern recognition receptor (PRR) agonists, cytokines, and oncolytics has been widely investigated to overcome “cold” tumor microenvironments (Nixon, N. et al. Current landscape of immunotherapy in the treatment of solid tumours, with future opportunities and challenges. Current oncology 25, 373-384 (2018); Aznar, M. A. et al. Immunotherapeutic effects of intratumoral nanoplexed poly I: C. Journal for Immunotherapy of Cancer 7, 1-16 (2019); Jelinek, I. et al. TLR3-specific double-stranded RNA oligonucleotide adjuvants induce dendritic cell cross-presentation, CTL responses, and antiviral protection. The Journal of Immunology 186, 2422-2429 (2011); Takeda, Y. et al. A TLR3-specific adjuvant relieves innate resistance to PD-L1blockade without cytokine toxicity in tumor vaccine immunotherapy. Cell reports 19, 1874-1887 (2017)). Several clinical-stage programs include TLR9-agonist idutolimod (CMP-001), TLR4-agonist tilsotolimod (IMO-2125), IL 12 inducer tavokinogene telseplasmid (TAVO), and oncolytic peptide KKWWKKW-Dip-K-NH2 (LTX-315). Interestingly, LTX-315 and GA share cationic properties conferred through constitutive lysine residues in the formulations. Our group also discovered that the cationic properties of GA enable its as a delivery vehicle for retaining other HIT-IT such as CpG and PolyI: C at the intratumoral site of injection (Pressnall, M. M. et al. Glatiramer acetate enhances tumor retention and innate activation of immunostimulants. International journal of pharmaceutics 605, 120812, doi: 10.1016/j.ijpharm.2021.120812 (2021)).

### Example 3: Immune Biomarker Expression in Copaxone® Treated Patients

The primary endpoint was adverse events associated with intratumoral/peritumoral injections of Copaxone®. Injection site reactions were observed in and around tumor tissues, like subcutaneous injections of Copaxone® reported in Multiple Sclerosis patients. Consistent reductions in Ki67, a marker of cell proliferation were observed.

Immune biomarkers were assessed in tumor biopsies and compared with those in the resected tumors after treatment. Conventional RNA sequencing and spatial protein profiling of 28 mostly immune-related targets (i.e., Nanostring GeoMx DSP) were used to assess immune markers at the transcriptional and protein levels. GeoMx illustrated upregulated markers of stimulatory immune activity (FIG. 1). These results were complimented by RNAseq gene expression analysis (FIG. 2), where genes were filtered for differentially expressed genes (|FC|<4) between pre-and post-samples for three patients. For patient 1, 6,355 genes were upregulated and 406 genes were downregulated post treatment. For patient 2,647 genes were upregulated and 75 genes were downregulated post treatment.

For patient 3,5375 genes were upregulated and 128 genes were downregulated post treatment. Gene Ontology (GO) enrichment analysis was performed to categorizes differential gene expressions into specific biological processes followed by Functional Annotation Clustering to group related GO terms. All three patients had an upregulation in genes involved in promoting an immune-rich tumor microenvironment such as NK cell activation, positive regulation of STAT protein, T-cell activation, humoral immune response, response to exogenous dsRNA, and B-cell proliferation and differentiation (FIG. 2). No biological processes were consistently found to be downregulated post treatment.

TABLE B
Clinical results from the GA Window of Opportunity trial.

| | | | | Gene |
|---|---|---|---|---|
| | Ki67 | | | expression |
| | results | | | (upregulated |
| Patient | (biopsy → | Copaxone | Injection Site | post- |
| Diagnosis | treated) | Injections | Reaction | treatment) |
| BCCa-Left | 65% → 60% | 2 | Grade 1-Mild | CD56, CD11c, |
| Nasal Ala | | | (itching/ | PD-L1, B7-H3 |
| | | | Pressure) | |
| BCCa-Right | 70% → 45% | 2 | Grade 1-Mild | VISTA, GITR |
| Post. Neck | | | (Burning | |
| | | | Sens.) | |
| SCCa-Right | 55% → 45% | 1 | None | CD8, CD4, |
| Cheek | | | | CD45, CD3, |
| | | | | OX401, GITR, |
| | | | | CD11c, PD-L1, |
| | | | | B7-H3, GZMB |
| BCCa-Right | 62% → 44% | 3 | 1^{st} inj. - Pain | CD56, CD45, |
| Flank | | | 2 hr No | CD8, CD3, |
| | | | further rxns | CTLA4, |
| | | | | LOX40L, |
| | | | | AG3, GZMB, |
| | | | | CD11c, CD20, |
| | | | | CD4, GITR, |
| | | | | STING, |
| | | | | PD-L1, |
| | | | | VISTA, Tim-3, |
| | | | | B7-H3 |
| Melanoma- | Pending | 2 | None | Pending |
| Left Scalp | | | | |
| Melanoma- | Pending | 2 | None | Pending |
| Mid Back | | | | |

The results in Table B demonstrate that GA treatment results in upregulation of immune markers, which are likely to be synergistic with or are targets for checkpoint inhibitors. For example, CD56 upregulated in 2 patients is a marker of Natural Killer T cells in the tumor, which correlate to better tumor outcomes when using immunotherapy. Another example, PD-L1 upregulated in 3 patients, is a target for three approved checkpoint inhibitors.

Intratumoral injections of Copaxone® were well tolerated with side effects at the injection site mirroring reported subcutaneous injection site reactions in Multiple Sclerosis patients. Immune markers including hallmarks of T cell recruitment and activation were upregulated in tumors. Our findings reveal that the local immunomodulatory effects of GA local to the intratumoral injection site in patients are safe and may suggest utility as human intratumoral immunotherapy.

### Example 4: Effects of GA and Immune Checkpoint Inhibitor Therapy in Cancer Models

### AT84 (Head and Neck Cancer)

AT84 cells were derived from a spontaneous sarcomatoid carcinoma in the oral mucosa of a C3H mouse and were graciously donated by Aldo Venuti (Regina Elena National Cancer Institute, Rome, Italy). Cells tested negative for interspecies contamination (Idexx BioResearch) and rodent pathogens (21 pathogen IMPACT I PCR profile), and negative for Mycoplasm contamination prior to animal studies (Lonza, MycoAlert test kit). Idexx CellCheck STR (short tandem repeat) profile: MCA-4-2: 20.3, 21.3; MCA-5-5: 15; MCA-6-4: 18, 19; MCA-6-7: 12; MCA-9-2: 15; MCA-12-1: 16; MCA-15-3: 25.3, 26.3; MCA-18-3: 16; MCA-X-1: 26, 27.

4% mannitol was prepared by dissolving D-mannitol (Fisher BioReagents® #M120, Fair Lawn, NJ) in molecular biology grade water (Corning #46-000, Manassas, VA) and sterile filtered through a 0.22 μm PES membrane filter (Millex®-GP #SLGPR33RS, Merck Millipore Ltd., Tullagreen, Carrigtwohill). 1 ml syringe stocks of 20 mg/mL GA (Copaxone®, TEVA Neuroscience, Inc., Overland Park, KS) were donated by The University of Kansas Medical Center. CpG ODN 1826 was purchased from InvivoGen (#tlrl-1826, San Diego, CA) and was resuspended in LAL water to obtain 3.17 mg/mL (500 μM) stocks following manufacture's protocol. All subsequent dilutions were performed using 4% mannitol. R4 polyplexes were prepared as previously described (Pressnall et al). Briefly, equal volumes of CpG was pipetted into a solution of GA while maintaining GA:CpG mass ratios of 4 (e.g. 6 mg/mL of GA into 1.5 mg/mL CpG), and the solution was mixed by quick pipetting for 30 s.

Wildtype C3H mice (Charles River Strain 025, 6-8 weeks old, 20-25 g) were used for in vivo AT84 tumor studies. Mice were anesthetized using 5% isoflurane in O₂ for approximately 5 min. One million AT84 cells in 50 μL cold DPBS (Gibco #14150, Life Technologies Corporation, Grand Island, NY) were injected subcutaneously into the floor of the mouth via an extra-oral route of C3H mice to procure orthotopic allograft tumors. Treatments began 12 days after AT84 cell injection. Under isoflurane anesthesia, mice were treated intratumorally with 4% mannitol vehicle, 300 μg GA alone, 75 μg CpG alone, or R4 (i.e. CpG in complex with GA) at 50 μL total injection volume administered every three days for 5 total treatments. 250 μg/100 μL isotype control (BioXCell #BP0089, Lebanon, NH) or anti-mouse PD-1 (BioXCell #BP0146) in In VivoPure pH 7.0 Dilution Buffer (BioXCell # IP0070) were also administered intraperitoneally every three days.

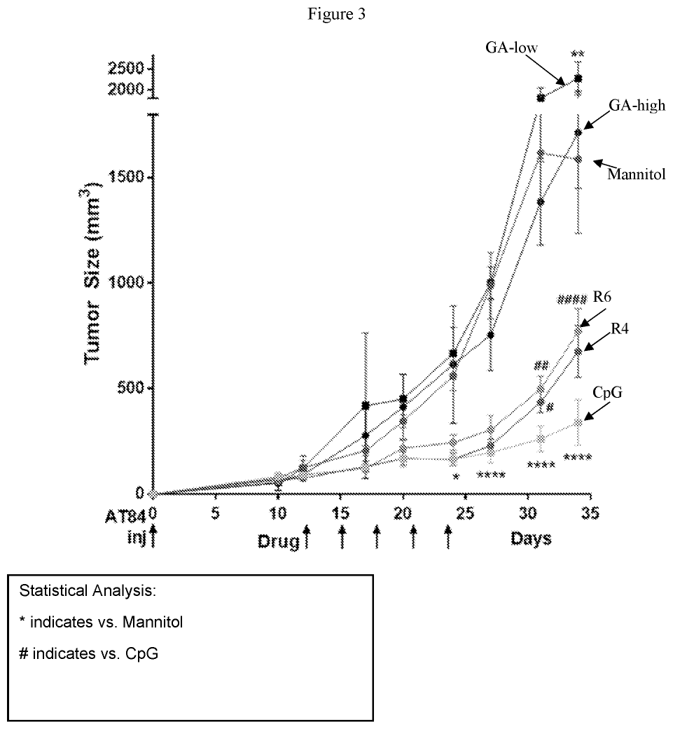

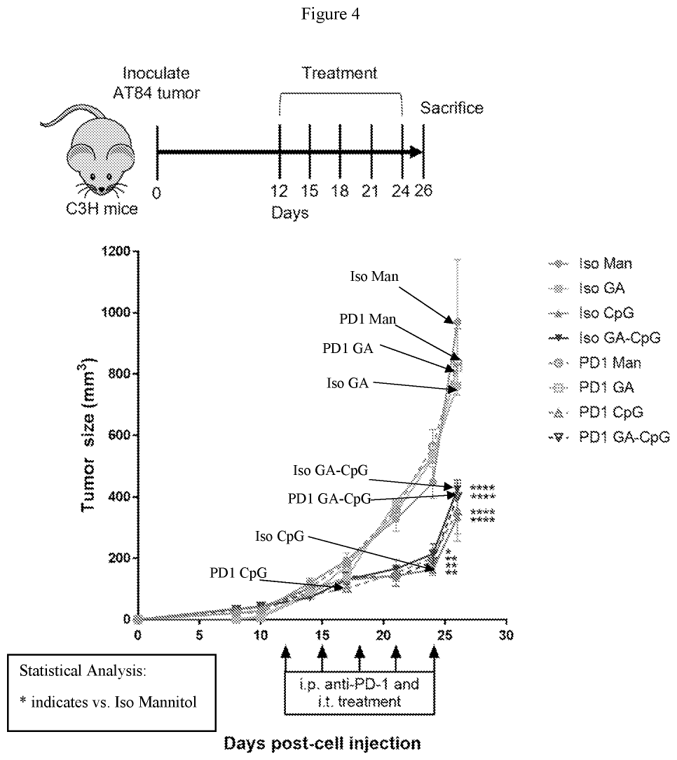

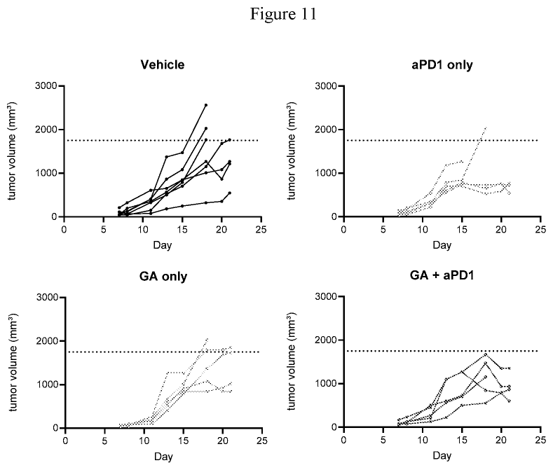

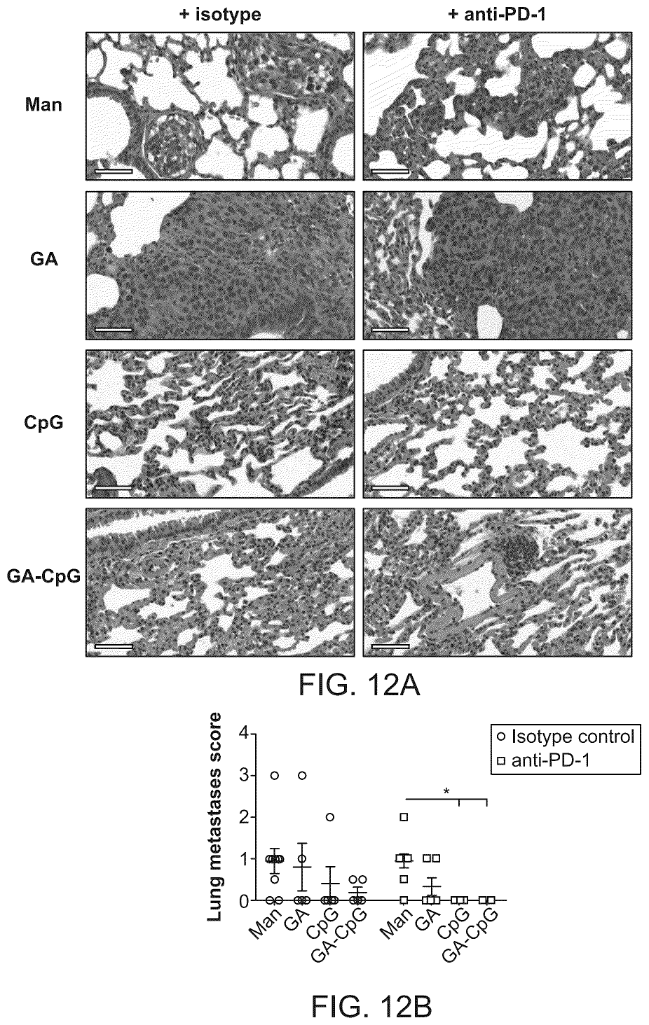

Results: A previous study (Pressnall et al.) demonstrated that low-dose GA (300 μg) led to significantly increased tumor growth in the AT84 model (FIG. 3) In the present study, both the CpG- and R4-treated groups had reduced tumor growth, but GA alone had no effect. However, the tumors in mice given anti-PD-1 antibodies grew similarly to the groups given the isotype control antibodies, suggesting that anti-PD-1 therapy alone did not influence tumor growth in the AT84 model (FIG. 4). However, anti-PD-1 therapy in AT84 tumor model reduced lung metastasis when administered in combination with intratumoral CpG or GA-CpG treatment (FIG. 12). Follow up analysis showed that C3H possess impaired T cell function and suggested that CpG's efficacy may have been exerted through an alternative mechanism.

### CT26 (Colorectal Cancer)

CT26 colon carcinoma cells were purchased from ATCC (#CRL-2638, Manassas, VA). Cells were cultured in RPMI-1640 medium with ATCC® modification (Gibco #A10491, Life Technologies Corporation, Grand Island, NY) supplemented with 10% FBS (Corning #35-010-CV, Woodland, CA), 100 U/mL penicillin, and 100 μg/mL streptomycin (HyClone #SV30010, South Logan, UT) in a humidified incubator at 37° C. and 5% CO₂.

Wildtype BALB/c mice (Charles River Strain 028; age 6-8 weeks and 20-25g) were used for in vivo CT26 tumor studies. Mice were anesthetized using 5% isoflurane in O₂for approximately 5 min. 100,000 CT26 cells in 100 μL cold DPBS/Matrigel (Corning #354248, Bedford, MA) (50:50) were subcutaneously implanted into the right flank of BALB/c mice to establish the murine colon adenocarcinoma cancer model. 100,000 cells CT26 cells were used as preliminary studies indicated that this concentration was best to study anti-PD-1 therapy. Anti-PD-1 treatment started 3 days after cell injection and intratumoral treatments started 6 days after cell injection.

Plasma was collected using K3EDTA tubes (Greiner Bio-One GmbH #450475, Abrera, Barcelona, Spain) via retroorbital bleeding 2 h after the first and fifth injections. Plasma cytokines were quantified using a mouse IL-6 DuoSet ELISA (R&D Systems #DY406, Minneapolis, MN) or a U-PLEX kit (Meso scale Diagnostics, LLC. #K15069L, Rockville, MD) following the manufacturer's protocol.

Tumor size was calculated using the equation, tumor volume (mm³)=0.52×(width)²×length, where length is the longer of two perpendicular dimensions. The final tumor size measurements were conducted by a researcher blinded to the study. At the end of the study, all animals were sacrificed, lungs were fixed with 10% neutral buffered formalin (Richard-Allan Scientific #5725, Kalamazoo, MI), and tumors were extracted. Tumor was bisected, and one half was frozen in OCT compound (Scigen Scientific #4585, Gardena, CA) for cryosectioning and staining. The other half was cut into small pieces (<5 mm) and stored in RNAlater™ stabilization solution (Invivogen™ #AM7021, Vilnius, Lithuania).

Staining tumor sections for immune cell markers: OCT embedded tumors were sectioned at 10 μm on a Shandon Cryotome FSE (Thermo Fisher Scientific, Kalamazoo, MI) onto Superfrost Plus Microscope Slides (Fisher Scientific #12-550-15, Pittsburgh, PA). The sections were fixed in 10% neutral buffered formalin, blocked with 10% goat serum (Gibco™ #16210064, Penrose, Auckland) in PBS (Fisher BioReagents™ #BP399, Fair Lawn, NJ), and stained with 5 μg/mL of primary antibodies in 5% goat serum in PBS buffer overnight at 4° C. Alexa Fluor® 488 anti-mouse CD8a Antibody (#100723), Alexa Fluor® 594 anti-CD11b antibody (#101254), Alexa Fluor® 647 anti-CD11c antibody (#117312),

Alexa Fluor® 488 anti-mouse CD3 antibody (#100210), Alexa Fluor® 594 anti-mouse CD4Antibody (#100446), and Alexa Fluor® 647 anti-mouse CD335 (NKp46) antibody (# 137628) were purchased from BioLegend (San Diego, CA). Corresponding isotype control antibodies (BioLegend #400525, 400661, 400924, 400625, and 400526) were also used as negative staining controls. Slides were then counterstained with 5 μg/mL Hoechst 33342 (Life technologies #H3570, Eugene, OR) for 15 minutes and mounted in SouthernBiotech™ Fluoromount-G™ Slide Mounting Medium (SouthernBioTech, Birmingham, AL). Images were acquired using an Olympus IX-81 inverted epifluorescence microscope at 10× magnification. The acquired images were compiled on Slidebook 6.0.

RNA isolation: RNA was isolated from the tumor as previously described. 22.83Briefly, tumor pieces were stored in RNAlater solution at room temperature for at least one day and then at 4° C. until RNA isolation. Tumor pieces were removed from RNAlater solution and homogenized in 10 mL TRIzol reagent (38% phenol, 0.8 M guanidine thiocyanate, 0.4 M ammonium thiocyanate, 0.1 M sodium acetate, 5% glycerol). Homogenate was divided into 1 mL, 100 μl BCP (Molecular Research Center #BP 151, Cincinnati, OH) was added, shaken vigorously 15 seconds and centrifuged 12,000×g, 10 minutes, 4° C. The top aqueous layer contains RNA, which was then precipitated with isopropanol (Fisher BioReagents™ #BP2618, Fair Lawn, NJ), centrifuged, and washed with 75% ethanol (Fisher BioReagents™ #BP2818) in DEPC-treated water. RNA was reconstituted in 1 mM sodium citrate pH 6.4 and stored at −80° C. RNA was quantified by Qubit™ using the RNA High Sensitivity Assay Kit (Invitrogen™ #Q32852, Thermo Fisher Scientific Corporation, Eugene, OR) and RNA quality was analyzed using the Agilent 2200 TapeStation (Waldbronn, Germany). Aside from two samples with a RNA integrity number (RIN) of 7.6 (pertaining to a sample from group Iso+GA and PD1+CpG), all of the other RNA samples had an RIN≤8.0, which is indicative of excellent quality.

RNA sequencing: The mRNA library preparation was performed with the NEBNext® Stranded mRNA library kit (New England Biolabs, Inc. #E7760, Ipswich, MA), tagged with a unique dual index sequence, and qPCR quantification of the library pool was performed before sequencing. Sequencing was performed on an Illumina NextSeq2000 P3 with single read 75 bp read lengths (KU Genome Sequencing Core). The reads were aligned with Mus musculus UCSC mm9 reference genome using RNA-Seq Alignment workflow (2.0.2, Illumina, San Diego, CA).

The transcript abundance estimates from Salmon were converted into gene-level count matrices using tximport (ver. 1.24.0).84 Count normalization was performed using DESeq2 (ver. 1.36.0), and the data was filtered by a lfcThreshold of 1 (i.e. absolute log2FoldChange>1; equivalent to a linear fold change of 2) and an adjusted p-value (padj) of <0.05 to obtain the differentially expressed genes.85 For heatmap generation, genes were filtered based on the differentially expressed genes of PD1+CpG treatment compared to Iso+Man treatment. A log2+1 transformation of normalized counts followed by scaling was applied to the data. The heatmap was generated using pheatmap (ver. 1.0.12). Gene ontology (GO) of the biological process, functional annotation clustering, and Kyoto Encyclopedia of Genes and Genomes (KEGG) pathway analysis of the differentially expressed genes was performed using Database for Annotation, Visualization and Integrated Discovery (DAVID) v2021.

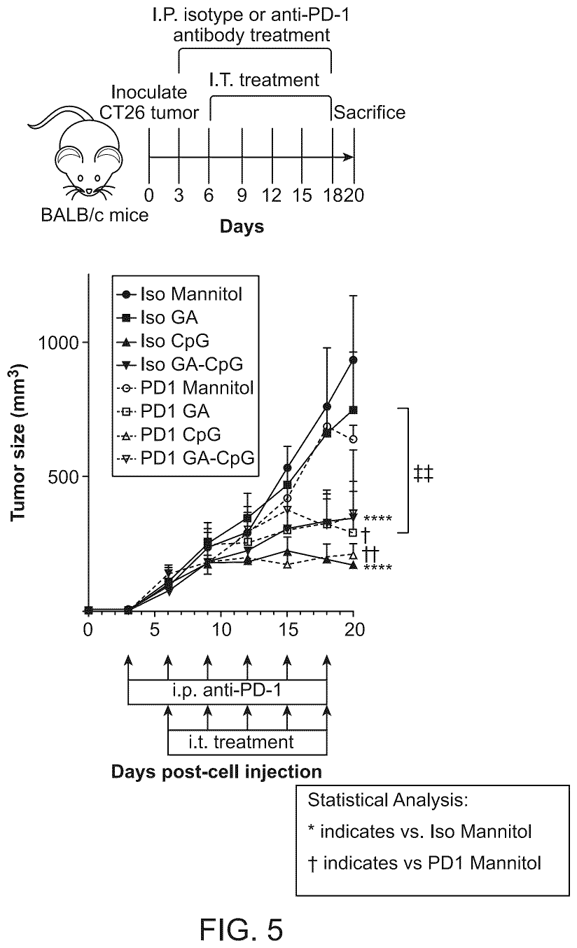

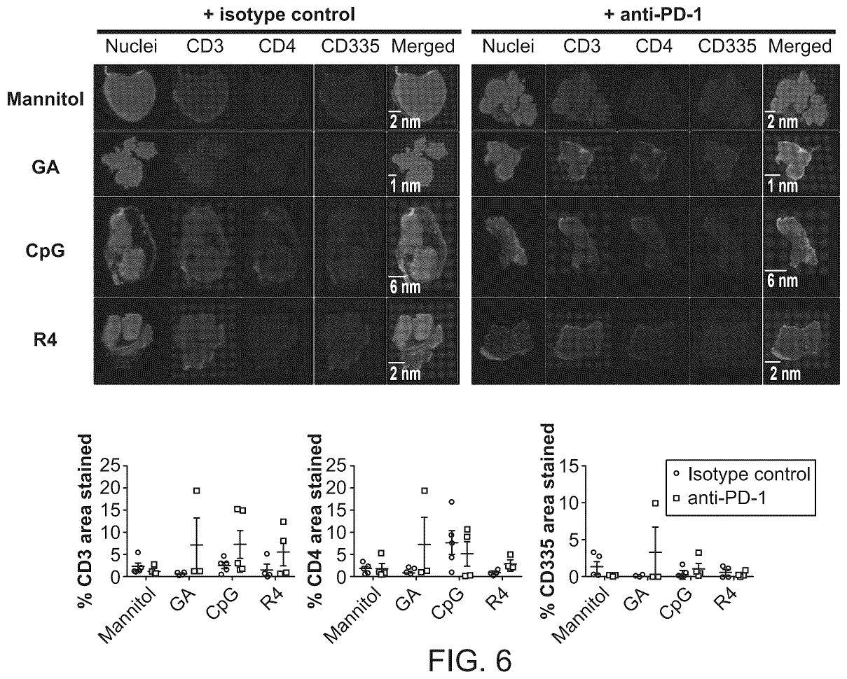

Results: Tumor growth curves showed that intratumoral treatments the combination of GA alone and anti-PD-1 resulted in a significant reduction in tumor growth that was not observed with monotherapy controls (FIG. 5). Significant changes to immunofluorescence markers were not present, but the best responding GA+anti-PD1 treated mouse exhibited markedly increased infiltration (FIG. 6). Increased CD3, CD4, and CD335 suggested enhanced helper T cell and NK cell presence.

RNA sequencing was also performed to determine the effects of intratumoral treatment on the tumor tissue. Gene Ontology (GO) of the biological process followed by functional annotation clustering and Kyoto Encyclopedia of Genes and Genomes (KEGG) pathway analysis was then performed on the differentially expressed genes to better elucidate the biological functions of these upregulated and downregulated genes. Generally, GA treatment had little discernable effect on gene expression in the tumors in this study design.

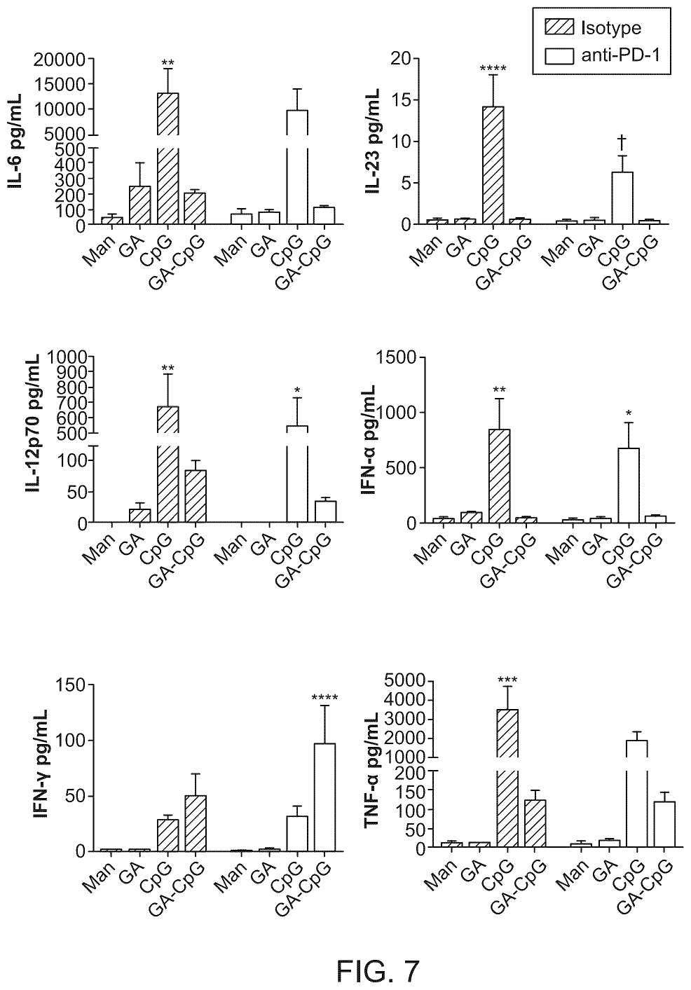

To evaluate the systemic effects of the treatments, we collected blood plasma from the mice 2 hours after first and last intratumoral treatment (i.e. on day 6 and 18). The combination of intratumoral GA and intraperitoneal anti-PD-1 treatment resulted in elevated systemic GM-CSF and IL-2 cytokine levels (FIG. 7) and demonstrated synergistic antitumor effects in the CT26 mouse tumor model. Moreover, tumors that responded most significantly to anti-PD-1 plus GA treatment showed increased markers of infiltration of CD⁴″ T cells and natural killer cells.

GA plus anti-PD1 combination reduced tumor growth in the CT26 model. The most responsive mouse from this treatment group possessed increased Th and NK cell infiltrates. Systemic IL-2 and GM-CSF were elevated by combination treatment, potentially contributing to therapeutic efficacy.

### EO771 (Breast Cancer)

GA was evaluated for its intratumoral efficacy and dose dependency in tumors alone and in combination with systemic anti-PD1 in the murine Triple negative breast cancer model EO771. EO771 cells were implanted to C57BL/6 mice on Day 0. In this study, two doses of intraperitoneal anti-PD1 (100 μg or 250 μg) and three doses of intratumoral GA (300 μg, 1 mg, 2 mg) were evaluated. Primary tumors were measured by caliper measurement. Tumor volumes were calculated according to the formula, tumor volume (mm³)=0.52×(width)² ×length, where length is the longer of two perpendicular dimensions.

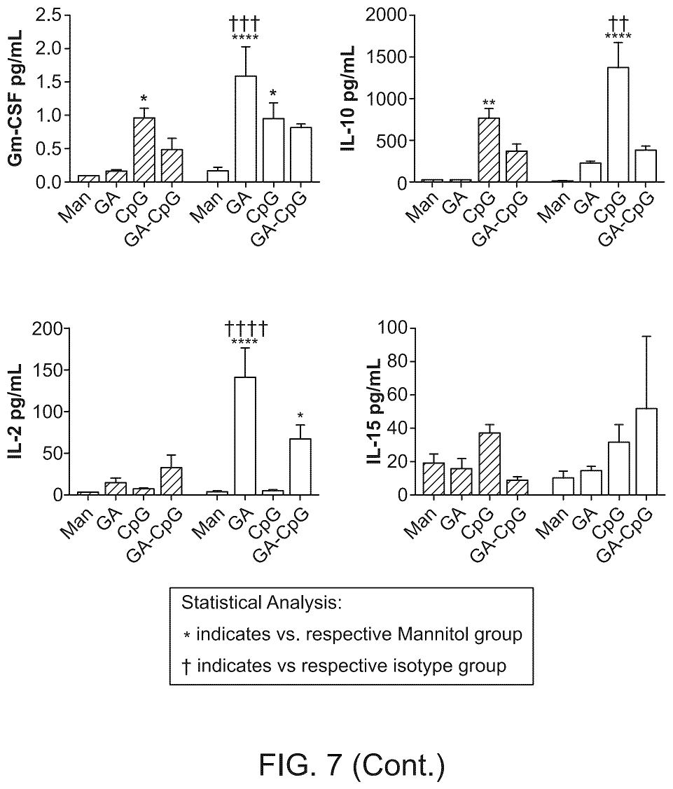

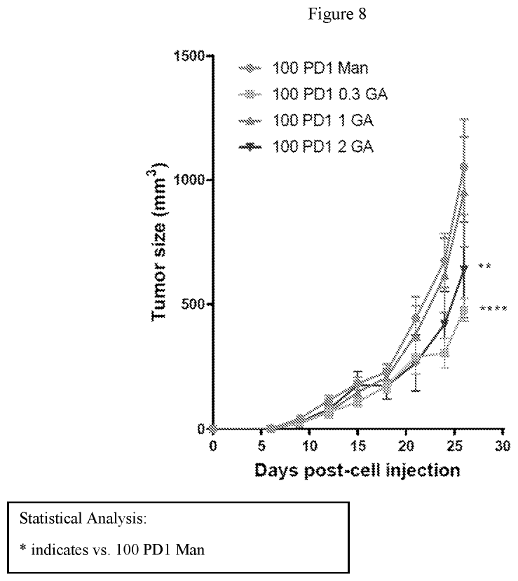

Results: 250 μg anti-PD1 dose was significantly more potent than 100 μg in the EO771 model. Among mice treated with 100 μg anti-PD1, 300 μg and 2 mg of intratumoral GA significantly reduced tumor growth (FIG. 8). In mice receiving 250 μg anti-PD1, additional benefit from intratumoral GA treatment was difficult to discern, but 2 mg of intratumoral GA led to larger tumor sizes than anti-PD1 treatment alone (FIG. 9). GA also does not induce systemic cytokines typically associated with cytokine release syndrome.

GA can synergize with anti-PD1 to limit tumor growth in the EO771 model.

### Cloudman M3 (Melanoma)

GA was evaluated for its intratumoral efficacy in tumors alone and in combination with systemic anti-PD1 in the murine melanoma tumor model Clone M3. 1×10⁶ Clone M3 cells were suspended in PBS and implanted in the left mammary fat pad of 4-5 week-old, male DBA/2N mice on Day 0. Mice were randomized into treatment groups when tumors reached mean volumes of 50-80 mm³ at Day 7.

Treatment groups consisted of vehicle control (n=7, 4% mannitol IT plus 10 mg/kg isotype control antibody IP), anti-PD1 alone (n=5, 4% mannitol IT plus 10 mg/kg anti-PD1 IP), GA alone (2 mg GA IT plus 10 mg/kg isotype control IP), or GA+anti-PD1 combination (n=5, 2 mg GA IT plus 10 mg/kg anti-PD1 IP). Intratumoral treatments were administered 5 times at a frequency of 3 times per week and 50 μL per injection beginning on Day 7. Intraperitoneal antibody was given beginning on Day 7 as well, but for a total of 3 times on a frequency of every 3-4 days.

Primary tumors were measured by caliper measurement. Tumor sizes were

calculated according to the formula (W²×L)/2. Animals were euthanized when any tumor diameter dimension exceeded 1.8 cm. The study was terminated when the 4^{th} mouse of the vehicle control group reached ethical euthanasia criteria.

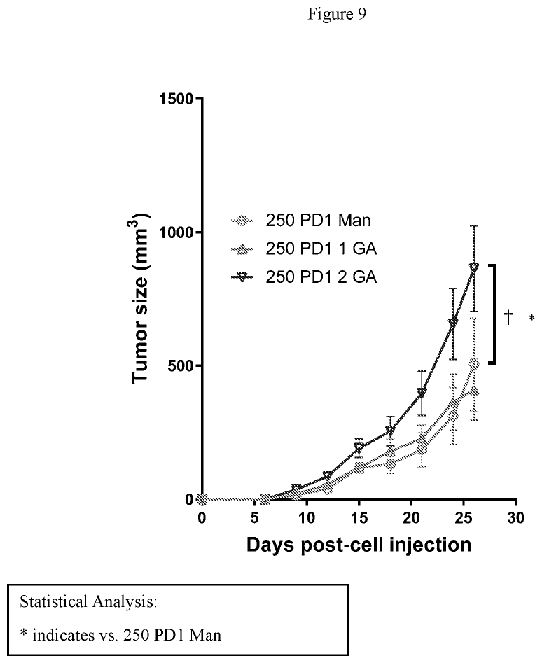

Results: Therapeutic efficacy was evaluated by assessing mouse survival against criteria of exceeding either 1750 mm³ tumor volume or 1.8 cm diameter (FIG. 10). Vehicle-treated were the most susceptible to M3 tumor growth throughout the study with three of seven mice surviving through 21 days. Mice receiving GA or anti-PD1 as monotherapies exhibited benefits to survival with three of five mice surviving in each group. Mice treated with both GA and anti-PD1 were most durable to M3 tumor progression with four of five mice surviving throughout the study.

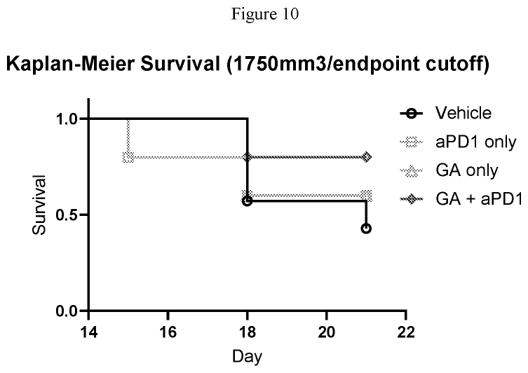

Interesting differences to individual growth curves were apparent among treatment groups (FIG. 11). Tumor response to anti-PDI monotherapy was bifurcated between responders (⅗ mice) and apparently complete nonresponders (⅖ mice). Combination treatment with intratumoral GA increased the proportion of responders to treatment (FIG. 10). Most mice treated with GA plus anti-PD1 exhibited tumors that began to reduce in size after treatment ceased on day 15.

GA plus anti-PD1 combination prolonged mouse survival. Most combination-treated mice exhibited shrinking tumor volumes after the conclusion of the treatment regimen.

### Example 5: GA in Combination with Anti-mPD-1 in the Syngeneic Melanoma Tumor Model CloneM3

Four experimental groups, one group containing seven and three groups each containing five female DBA/2N mice after randomization. On Day 0, 1.0×10⁶ CloneM3 (Z1) tumor cells in 100 μl PBS were implanted into the left mammary fat pad of each mouse. Tumor growth was monitored by caliper measurement throughout the study.

After animals had been randomized on Day 7, treatments were initiated on the same day. GA was administered intratumorally at a dose of 50 μl/mouse 5× on Days 7, 9, 11, 13 and 15 in combination with either Isotype Control at 10 mg/kg given intraperitoneally 3× on Days 7, 11 and 15 (Group 3), or anti-mPD-1 at 10 mg/kg given intraperitoneally 3× on Days 7, 11 and 15 (Group 4). Both groups were compared to Vehicle Control administered intratumorally at a dose of 50 μl/mouse 5× on Days 7, 9, 11, 13 and 15 in combination with Isotype Control at 10 mg/kg given intraperitoneally 3× on Days 7, 11 and 15 (Group 1). Also evaluated was the Vehicle Control administered intratumorally at a dose of 50 μl/mouse 5× on Days 7, 9, 11, 13 and 15 in combination with anti-mPD-1 at 10 mg/kg given intraperitoneally 3× on Days 7, 11 and 15 (Group 2).

During the course of the study, several animals of the cohorts had to be euthanized due to ethical abortion criteria (mostly tumor size) prior to study end without performing necropsies. After the fourth animal of Group 3 reached ethical abortion criteria on Day 26, this part of the study was terminated, all remaining animals were sacrificed, and a necropsy was performed.

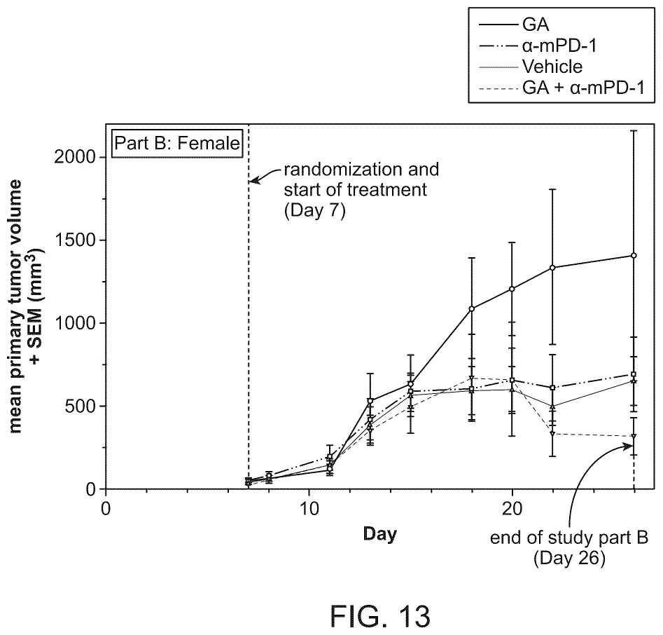

In summary, after Day 20, a reduction in tumor growth was observed for GA in combination with anti-mPD-1 (Group 4) compared to anti-mPD-1 alone (Group 2). See FIG. 13.

### Example 6: GA in Combination with Anti-mPD-L1 in the Syngeneic CT26wt Tumor Model

Each experimental group contained eight female BALB/c mice after randomization. On Day 0, 0.5×10⁶ CT26wt murine colon adenocarcinoma tumor cells in 100 μl PBS were implanted into the left mammary fat pad of each mouse.

After animals had been randomized on Day 6, treatments were initiated on the same day. Test compound GA was administered at 1 mg/25 μl/mouse intratumorally (i.t.) for 5 times 3 days apart on Days 6, 9, 12, 15 and 18 either alone (in combination with Isotype Control) or in combination with anti-mPD-L1, administered at 10 mg/kg intraperitoneally (i.p.) 3 times 3-4 days apart on Days 6, 9 and 13, and was evaluated versus anti-mPD-L1alone (each in combination with Vehicle Control). All groups were evaluated versus the Control, which were animals that had been treated with 25 μl/mouse Vehicle Control (4% mannitol) i.t. in combination with 10 mg/kg Isotype Control i.p.

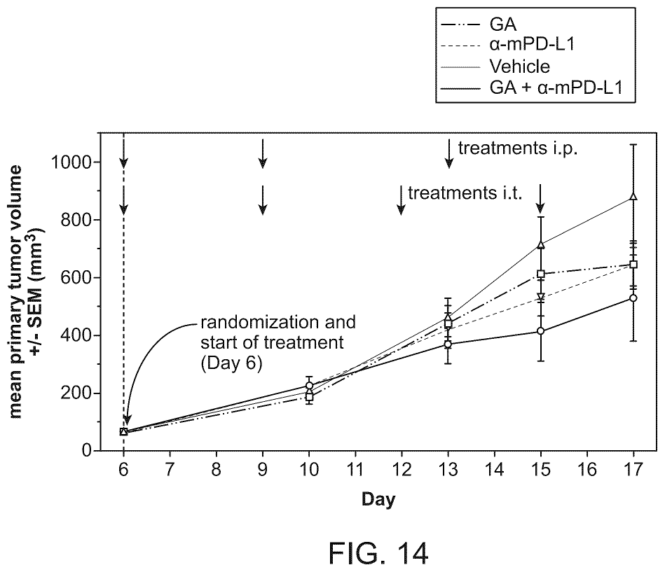

During the course of the study, individual animals were either euthanized prior to study end without performing a necropsy, after they had reached humane endpoint, or were found dead. On Day 45, the study was terminated and all remaining animals were euthanized without performing a necropsy. As shown in FIG. 14, GA in combination with anti-PD-L1 further decreased tumor growth in the CT26wt model relative to anti-PD-L1 therapy alone. Repeat PDL1 experiments demonstrated abscopal effect.

### Example 7: GA in Combination with Anti-CTLA4 in the Syngeneic Tumor Models

Tumor cells are implanted to C57BL/6 mice on Day 0. After animals have been randomized on Day 6, treatments are initiated on the same day. Test compound GA is administered at 1 mg/25 μl/mouse intratumorally (i.t.) for 5 times 3 days apart on Days 6, 9, 12, 15 and 18 either alone (in combination with Isotype Control) or in combination with anti-mCTLA-4, each administered at 10 mg/kg intraperitoneally (i.p.) 3 times 3-4 days apart on Days 6, 9 and 13, and is evaluated versus anti-mCTLA-4 alone (each in combination with Vehicle Control). All groups are evaluated versus the Control, which are animals treated with 25 μl/mouse Vehicle Control (4% mannitol) i.t. in combination with 10 mg/kg Isotype Control i.p.

During the course of the study, individual animals are either euthanized prior to study end without performing a necropsy, after they reach humane endpoint, or are found dead. On Day 45, the study will be terminated and all remaining animals are euthanized without performing a necropsy.

It is anticipated that GA will synergize with anti-CTLA4 to further limit tumor growth and/or improve survival in one or more tumor models compared to anti-CTLA4 therapy alone.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A method for treating cancer or inhibiting tumor growth in a patient in need thereof comprising administering to the patient an effective amount of glatiramer acetate (GA) and an effective amount of an immune checkpoint inhibitor.

2. The method of claim 1, wherein glatiramer acetate is administered intratumorally and/or is complexed with CpG oligodeoxynucleotides.

3. The method of claim 1, wherein the immune checkpoint inhibitor is administered intramuscularly, intraperitoneally, subcutaneously, intravenously, or systemically.

4. The method of claim 1, wherein the glatiramer acetate is administered separately, simultaneously, or sequentially with the immune checkpoint inhibitor.

5. The method of claim 1, wherein the cancer is adrenal cancer, bladder cancer, bone cancer, brain cancer, breast cancer, carcinoma, cervical cancer, colorectal cancer, uterine cancers, ear, nose and throat (ENT) cancers, endometrial cancer, esophageal cancer, gastrointestinal cancer, head and neck cancer, intestinal cancer, kidney cancer, larynx cancer, liver cancer, lung cancer, melanoma, mesothelioma, nasopharynx cancer, neuroblastoma, oral cancer, ovarian cancer, pancreatic cancer, penile cancer, pharynx cancer, prostate cancer, teratomas, testicular cancer, thyroid cancer, vaginal cancers, or vascular tumors.

6. The method of claim 1, wherein the immune checkpoint inhibitor comprises one or more of an anti-PD-1 antibody, an anti-PD-L1 antibody, an anti-PD-L2 antibody, an anti-CTLA-4 antibody, an anti-TIM3 antibody, an anti-CD73 antibody, an or an anti-LAG-3 antibody.

7. The method of claim 1, wherein the immune checkpoint inhibitor comprises one or more of pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IBI308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, or BMS-986189.

8. The method of claim 1, wherein the patient is human.

9. The method of claim 1, wherein the glatiramer acetate comprises a heterogenous mixture of copolymers consisting of L-glutamic acid, L-alanine, L-tyrosine, and L-lysine in a molar ratio of 0.14:0.43:0.09:0.34.

10. The method of claim 1, wherein the glatiramer acetate has a molecular weight ranging from 2,500 to 20,000 Da, or an average molecular weight ranging from 5,000 to 9,000 Da.

11. A kit comprising glatiramer acetate, an immune checkpoint inhibitor, and instructions for treating cancer.

12. The kit of claim 11, wherein the glatiramer acetate is complexed with CpG oligodeoxynucleotides.
